(19) United States
(12) Patent Application Publication
(10) Pub. No.: US 2011/0021973 A1
(43) Pub. Date: Jan. 27, 2011

(54) FORMULATIONS FOR COSMETIC AND WOUND CARE TREATMENTS WITH PHOTOSENSITIZERS AS FLUORESCENT MARKERS

(75) Inventors: Wolfgang Neuberger, Labuan (MY); Susanna Gräfe, Jena (DE); Nikolay E. Nifantiev, Moscow (RU)

Correspondence Address: MCCARTER & ENGLISH, LLP HARTFORD;CITYPLACE I, 185 ASYLUM STREET, HARTFORD, CT, 06103, US

(73) Assignee: CeramOptec Industries, Inc.

(21) Appl. No.: 12/896,614

(22) Filed: Oct. 1, 2010

Related U.S. Application Data
(63) Continuation of application No. 12/006,911, filed on Jan. 7, 2008.
(60) Provisional application No. 60/881,107, filed on Jan. 18, 2007.

(51) Int. Cl. A61M 37/00

(52) U.S. Cl. 604/20

(57) ABSTRACT

Photoactive materials, such as photosensitizers, are used as fluorescent markers for in vivo detection of the distribution of the injected filler material during cosmetic treatments. In one preferred embodiment, liposomal formulated temoporfin is used, as the photoactive component, in very small concentrations along with fillers for cosmetic and wound healing applications. Fillers, which can be used in the invention, include collagen, hyaluronic acids and other synthetic or natural products which are generally used in wound healing, scar reduction and other such medical applications. In a preferred embodiment, the formulated photosensitizer is coupled to the filler so that tracking is possible over longer periods of time A liposomal formulated photosensitizer is injected with the fillers into the treatment area, and is irradiated with laser light shortly after injection. The emitted fluorescence is measured by a special non-invasive device. Thereby it is possible to monitor the injection site and the distribution of the injected solution around the injection site. When irradiated with laser or other light source, the fluorescence of the photosensitizer is detected using a fluorescence detector, which permits tracking the filler at injection site and in the injection volume.

### DOMESTIC PRIORITY UNDER 35 USC 119(c)

This application claims benefit of U.S. Provisional Application Ser. No. 60/881,107 filed Jan. 18, 2007, which is incorporate by reference herein.

### BACKGROUND OF THE INVENTION

1. Field of the Invention

Present invention relates to fluorescent markers in general and more specifically for use of Photosensitizer in biological filler as fluorescent marker, to trace the success of different types of fillers used for tissue repair/augmentation, and other cosmetic applications.

2. Invention Disclosure Statement

In cosmetic surgery and wound healing treatments natural or synthetic biological materials are used. To treat third degree burn injuries, deep cut, or even for cosmetic correction of skin imperfection biological materials called fillers are used widely. The fillers include collagen, hyaluronic acid or others such synthetic materials.

The protein collagen is the main substance of connective tissue and is present in humans. In mammals collagen is the most abundant protein. Collagen gives many different organs and tissues support and elastic properties. It has been found in many different tissues and organs like bones, tendons, (hyaline) cartilage, blood vessels, teeth, cornea, skin, etc. It prevents organs/tissues from tearing or losing their functional shape when they are exposed to sudden and wild movements.

Collagens are fibrous protein composed of amino acids. The most abundant amino acids are glycine, proline and hydroxyproline. General collagen structure consists of three polypeptides, each of which is a left-handed helix, intertwined into a right-handed triple helix. Human body is mainly composed of collagen type I, II, and III, however many other types are also present.

Collagen is a natural biomaterial commonly used in tissue engineering and repair; it has negligible immune rejection and excellent biocompatibility. But unprocessed collagen is mechanically weak and vulnerable to chemical and enzymatic attacks that limits its use.

Collagen can be cross-linked to increase its molecular stability and mechanical properties. The most basic mode of action is the covalent intermolecular cross-link formation between collagen fibrils. Cross-linking improves strengths, resorption rate and biocompatibility of the scaffold.

Hyaluronic acid (non-animal stabilized hyaluronic acid) is also widely used as a filler of natural origin for a variety of cosmetic applications. It is one of the chief components of the extracellular matrix. It is an FDA approved product for filling soft tissue defects such as facial wrinkles, scars and other skin imperfections for aesthetic purposes. Hyaluronic acid is a substance found naturally in the human body. It is hydrophilic in nature, hence acts as a sponge to absorb water and provide long lasting results when used as fillers with low risk of allergic reaction. Its high viscoelastic character has been used to supplement the lubricant in arthritic joints.

The use of fluorescence imaging for in vivo and ex vivo characterization of biological materials has been well established for several decades based on the specific localization of administered fluorescent molecules in tissue or cell structures. Techniques frequently used clinically in vivo include fluorescein angiography to image the retinal vasculature, and for guidance of surgical resections.

Photosensitizer fluorescent markers are used in prior art for detection of abnormal cells in vivo. Photosensitizer used in photodynamic therapy is also used in photodynamic detection of abnormal cells. In this technique a photosensitive material, which has an affinity to tumors and emits fluorescence when excited by light, is first administered to the tumor as a fluorescence diagnosis agent. Then an excitation light having a wavelength; in the exciting wavelength range of the photosensitive material is projected onto the tumor to cause the fluorescence of the diagnosis agent, collected in the tumor. The tumor is diagnosed on the basis of an image which is formed by the fluorescence and shows the location and the area of infiltration of the diseased part.

The present invention provides formulations and a method of using photosensitizers together with biological fillers as fluorescent markers in cosmetic and wound care applications, for detecting injection site and filler distribution in the injection area, among other benefits, without causing a cytotoxic effect on the filler. Certain photosensitizer with fluorescent properties is chosen as a preferred embodiment.

### OBJECTIVES AND BRIEF SUMMARY OF THE INVENTION

It is an objective of the present invention, to use a fluorescent marker for in vivo detection of filler and related additives in cosmetic applications and wound healing treatments.

It is another objective of the present invention, to use photosensitizer as the fluorescent marker.

It is also an objective of the present invention to use photosensitizer as a fluorescent marker in cellulite treatment and other skin deep cosmetic applications.

Briefly stated, in the present invention photosensitizers are used as fluorescent markers for in vivo detection of the distribution of the injected filler material during cosmetic treatments. In one preferred embodiment, liposomal formulated temoporfin is used, as the photosensitive component, in very small concentrations along with fillers for cosmetic and wound healing applications. Fillers, which can be used in the invention, include collagen, hyaluronic acids and other synthetic or natural products which are generally used in wound healing, scar reduction and other such medical applications. In another preferred embodiment the formulated photosensitizer is coupled to the filler so that tracking of the filler is possible over longer periods of time. A liposomal formulated photosensitizer is injected with the fillers into the treatment area, and is irradiated with laser light shortly after injection. The emitted fluorescence is measured by a special non-invasive device. Thereby it is possible to monitor the injection site and the distribution of the injected solution around the injection site. When irradiated with laser or other light source, the fluorescence of the photosensitizer is detected using a fluorescence detector, which permits to tracking the filler at injection site and in the injection volume.

The above and other objects, features and advantages of the present invention will become apparent from the following description read in conjunction with the accompanying drawings.

### BRIEF DESCRIPTION OF FIGURES

FIG. 1—structure of a hydrophobic photosensitizer useful in a preferred embodiment of the present invention

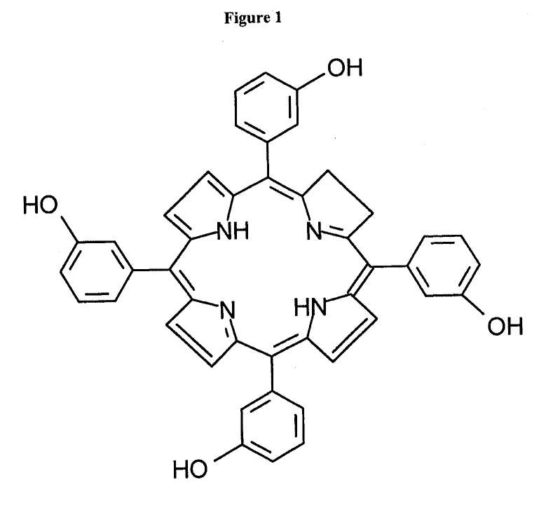

### DETAILED DESCRIPTION OF PREFERRED EMBODIMENTS

A photosensitizer containing formulation is used in fluorescent or fluoroscopic detection for in vivo diagnosis of cancerous cells internally and also in superficial tumors of the skins. Photosensitizer is applied either topically or systemically which accumulates selectively in tumor cells. By irradiation with light with the proper excitation wavelength the photosensitizer molecules are induced to fluoresce. The emitted fluorescence light can be displayed by an optical system and enables visualizing the localization of the tumor.

In one embodiment, biological fillers are used in combination with photosensitizer for cosmetic applications, like tissue repair or augmentation, wherein the photosensitizer is used as fluorescent marker to detect the injection places and distribution of the injected solution in tissue around the treatment site.

The variation in the injection volume and ineffective delivery of drug concentration to treatment site is common problems in cosmetic application. This is due to various reasons like needle system employed for drug delivery, and loss of drug due to bleeding at the site of multiple injections. In the present invention the fluorescent marker is employed to trace the injected volume at the site and to delivery required volume thus maintain the consistence required in cosmetic applications.

The biological fillers that can be used in the invention include collagen, hyaluronic acid and other biocompatible materials. The photosensitizer is combined with the biological filler and the formulation is injected into the treatment site. The injection volume is traced by fluorescence so that efficiency of treatment and reproducibility of injection volumes is consistent through out the procedure. The successful application of the filler can then be monitored by irradiating the injected sites with appropriate light to induce fluorescence, which is carefully measured.

Collagen is a natural biomaterial used for tissue reconstruction in third degree burns, wounds and for cosmetic application. Collagen that is currently used has a few drawbacks like being mechanically weak, having low stability, swelling rapidly in water and being susceptible to chemical and enzymatic attack when implanted.

Collagen sponges like Gentacoll, Kollagen Resorb (Resorba GmbH) and Collagen Fibrils (Collagen Matrix Inc)] were used with PS for healing different types of wound and to improve cosmetic appearance of the skin surface. In case of wrinkle reduction on face and neck region injectable formulation of collagen is required for best cosmetic effect. Collagen is injected through tiny needle just below the surface of the skin to smooth wrinkles. Examples of injectable collagen are Zyplast and Zyderm (produced by Inamed Aesthetics Inc. USA). Zyplast and Zyderm are derived from the collagen of cow skin.

As a variation, Hyaluronic acid (non-animal stabilized hyaluronic acid) can be used as a dermal filler to correct wrinkles, scars and other skin deformities for aesthetic purposes. Hyaluronic acid is a substance found naturally in a human body. It is hydrophilic in nature, hence acts as a sponge to absorb water and provide long lasting results when used as filler with low risk of allergic reaction.

Photosensitizers themselves can be used as a fluorescent marker. Temoporfin, which has been used as an exogenous photoactive agent for PDT in a wide field of cancer treatment, is a useful example. Besides its high affinity to hyperplastic tissue and its high phototoxicity at low activation energies, mTHPC, when illuminated by near ultraviolet light of the proper wavelength, also exhibits a strong fluorescence, which can be exploited for visualization of cells under investigation.

In the present invention hydrophobic photosensitizers are integrated within the lipid bilayer of special liposomes. A liposomal formulation is prepared in general by dissolving a hydrophobic photosensitizer and the synthetic phospholipids in suitable alcoholic solvents. The preferable synthetic phospholipids include dipalmitoyl phosphatidyl choline (DPPC), dimyristoyl phosphatidyl choline (DMPC), dipalmitoyl phosphatidyl glycerol (DPPG), dimyristoyl phosphatidyl glycerol (DMPG) and when pegylation is desirable, pegylated distearoyl phosphatidyl ethanolamine (DSPEG). A hydrophobic photosensitizer is selected from a group consisting of chlorins and bacteriochlorins; of which temoporfin is an example.

This solution is dried under vacuum, causing the alcoholic solvent to evaporate. The solid residue, which is obtained, is homogenized by dispersing in a monosaccharide solution. Then the solution is freeze-dried for storage and reconstituted in suitable aqueous solution for administration.

In various embodiments, a temoporfin composition may be injected, ingested, applied topically, transdermally, or subcutaneously. After administration, the photosensitizer composition accumulates, in a target tissue. The selected target site, requiring diagnosis is exposed to light of the proper wavelength causing fluorescence to render a diagnosis. The liposomal formulated temoporfin is administered in low doses, for example, 30-450 ng/ml, which are effective to achieve the desired diagnostic effect. Such doses may vary widely depending upon the particular compounds employed in the composition, the organs or tissues to be examined, the equipment employed in the clinical procedure, the efficacy of the treatment achieved, and the like. These compositions contain an effective amount of the compound(s), along with drug carriers and excipients appropriate for the type of administration.

In another embodiment, the photosensitizer fluorescent agents may be formulated as micelles, microcapsules, or other microparticles. These formulations may enhance delivery, localization, target specificity, administration, etc.

The present invention is further illustrated by the following examples, but is not limited thereby.

### Example 1

### Uses of Liposomal Temoporfin as Marker with Collagen as Filler for Wrinkle Removal

A low concentration of liposomal formulated temoporfin (3-5 μg/ml mTHPC) is injected with collagen into a treatment site. The liposome formulation of hydrophobic temoporfin is beneficial as it increases water solubility. After injection the site is illuminated with suitable excitation wavelength, which generally is different from the photosensitizer's main absorption peak, thus avoiding cytotoxic damages to cells. For temoporfin (mTHPC) its spectrum has an excitation maximum at 417 nm while the main absorption peak is at 652 nm. The photosensitizer used here serves as a visual marker indicating the success of injected collagen material.

### Example 2

### Uses of Temoporfin as Marker to Study the Photochemical Cross-Linking of Collagen

To trace the photochemical cross-linking of collagen using photoactive compound a small amount of liposomal formulation containing a low concentration (3-15 μg/ml) of temoporfin (mTHPC) is used with collagen. In this case temoporfin is used as marker as well as photo therapeutic compound. The spectral character of temoporfin shows excitation maxima at 417 nm and an activating peak at 652 nm. Photochemical cross-linking of the collagen can be followed by monitoring the fluorescence of temoporfin excited by 417 nm.

### Pepsin Promoted Collagen Gel Degradation Study Using Temoporfin Marker Material:

3 batches of collagen gels, collagen content 9.37 mg/ml Collagen I rat tail (2 test plates containing 4 samples)

TABLE 1
Collagen Gel Batch Formulation

| | | | Irradiation 652 nm |
|---|---|---|---|
| batch | Consistence | mTHPC (μg/ml) | (J/cm²) |
| 1 | Gel | 0 | 10 |
| 2 | Gel | 5 | 10 |
| 3 | Gel | 5 | — |

Batches 1, 2 and 3 are prepared, each containing a 15 ml solution of commercially available 1 MT Pepsin (0.5 U/mg; Mr˜36.000) in 100 MT 0.1 M HCl tempered to 37° C. Collagen gel batches 1, 2 and 3 are added to respective batches.

Batches 1, 2 and 3 are then incubated on a shaker at 37° C. and 100 rpm until the collagen gel is completely resolved (5 to 6 recurrences per sample) (Table 1).

Results from the above study, Table 2, showed a detectable increase of cross-linking of collagen using photosensitizer and irradiation. The level of cross-linking was light dependent.

TABLE 2
Decomposition Time of Collagen Gel Batches in Pepsin Solution

| Batch | Description | Time (min) |
|---|---|---|
| 1 | Irradiation | 5 |
| 2 | Photosensitizer + irradiation | 104 |
| 3 | Photosensitizer | 65 |

### Example 3

### Uses of Temoporfin as Marker with Hyaluronic Acid as Dermal Filler

Medical devices composed of hyaluronic acid and liposomal formulated mTHPC are used as dermal fillers. In an example, 1 ml (20 mg/ml injecting solution) of hyaluronic acid is mixed with a liposomal formulated temoporfin, wherein, temoporfin is present in a low concentration (3-10 μg/ml) for tracking the hyaluronic acid in vivo. This formulation is water soluble and is further diluted with water to get the final injecting solution. After injecting the formulation having hyaluronic acid with a liposomal formulated temoporfin, the site is illuminated with suitable excitation wavelength which is not generally identical with main absorption (activation) peak of the temoporfin, thus avoiding cytotoxic damages to cells. The injected site may also be covered with a light blocking plaster or plastic to protect the skin area from phototoxic effect of light exposure for few days. The light was incident uniformly over the treated area and the emitted fluorescence was collected from tissues. A fluorescent fiber spectrometer with deep light penetration is used for fluorescence detection. This then is used to track the placement of the hyaluronic acid solution as it diffuses in the treated tissue.

### Example 4

### Uses of Temoporfin as Marker with Hyaluronic Acid while Lubricating the Arthritic Joints

Hyaluronic acid was mixed with liposomal formulated temoporfin, wherein, temoporfin was present in a low concentration (3-54 ml) for tracking the hyaluronic acid in-vivo. Hyaluronic acid with a liposomal formulated temoporfin was injected into the arthritic joints; the site or sites were illuminated with a 417 nm excitation wavelength which is different from the main absorption/activation wavelength (−652 nm) of the temoporfin, thus avoiding cytotoxic damages to cells. The light was incident uniformly over the treated area and the emitted fluorescence was collected from tissues. This then was used to track the replacement of synovial fluid within the joint.

Having described preferred embodiments of the invention with reference to the accompanying drawings, it is to be understood that the invention is not limited to the precise embodiments, and that various changes and modifications may be effected therein by skilled in the art without departing from the scope or spirit of the invention as defined in the appended claims.

1-13. (canceled)

14. A method for monitoring the distribution of biocompatible filler material injected to a treatment site during tissue repair/augmentation or other cosmetic applications comprising the steps of:
injecting at a treatment area a formulation comprising a biocompatible filler material and a photoactive material which can fluoresce, wherein said photoactive material is a liposomal formulation of hydrophobic photosensitizers; and
irradiating the treatment area with light at a wavelength that will cause the photoactive material to fluoresce.

15. The method according to claim 14, wherein the injected formulation further comprises a carrier material to facilitate said formulation to arrive at a selected treatment site.

16. The method according to claim 14, wherein said biocompatible filler material is selected from the group consisting of collagen and hyaluronic acid.

17. The method according to claim 16, wherein said photoactive material is selected from the group consisting of chlorins and bacteriochlorins.

18. The method according to claim 17, wherein said photoactive material is temoporfin.

19. The method according to claim 14, wherein said photoactive material is encapsulated in a liposomal formulation is produced from synthetic phospholipids.

20. The method according to claim 19, wherein said synthetic phospholipids are selected from the group consisting of dipalmitoyl phosphatidyl choline (DPPC), dimyristoyl phosphatidyl choline (DMPC), dipalmitoyl phosphatidyl glycerol (DPPG), dimyristoyl phosphatidyl glycerol (DMPG), poly (ethylene glycol)-linked phospholipids and combinations of these materials.

21. The method according to claim 19, wherein said liposomal formulation includes at least one poly(ethylene glycol)-linked phospholipid.

22. The method according to claim 1, further comprising the step of measuring the emitted fluorescence from the photoactive material using a non-invasive device.

23. The formulation according to claim 18, wherein the concentration of temoporfin is between 0.03 to 10 μg/ml.
